(19) United States
(12) Patent Application Publication
(10) Pub. No.: US 2005/0106253 A1
(43) Pub. Date: May 19, 2005

(54) Pharmaceutical pellets comprising tamsulosin

(76) Inventor: Johannes J. Platteeuw, s'Hertogenbosch (NL)

Correspondence Address: MARK R. BUSCHER, P.O. BOX 161, CATHARPIN, VA, 20143, US

(21) Appl. No.: 10/705,996

(22) Filed: Nov. 13, 2003

(51) Int. Cl. A61K 9/24; A61K 9/14

(52) U.S. Cl. 424/471

(57) ABSTRACT

Tamsulosin pellets having an advantageous release profile are formed. The pellets have an enteric coating and release less than 25% of the tamsulosin in two hours in SGF.

### BACKGROUND OF THE INVENTION

The present invention relates to coated tamsulosin pellets and to unit dosage forms made therefrom.

Tamsulosin is the common name for 5-[2-[[2-(2-ethoxyphenoxy)ethyl]amino]propyl]-2-methoxy-benzenesulfonamide of the formula (1). 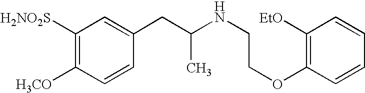
It is disclosed in EP 34432 and U.S. Pat. No. 4,731,478 as a pharmaceutically active substance having alpha-adrenergic blocking activity that is useful for treatment of cardiac insufficiencies and benign prostatic hyperplasia.

(R)-tamsulosin hydrochloride is marketed under various trade names, including FLOMAX® (Boehringer Ingelheim) in the U.S., HARNAL® (Yamanouchi) in Japan and OMNIC® (Yamanouchi) in Europe, for treatment of symptoms of benign prostatic hyperplasia (also known as BPH) such as urinary volume and frequency problems. The approved drug products include a capsule dosage form for oral administration that comprises 0.4 mg of the tamsulosin hydrochloride. The capsule provides controlled release of the tamsulosin and is a once daily dosage form, although two capsules can be used if needed; i.e. a maximum single daily administration of 0.8 mg. U.S. Pat. No. 4,772,475 is listed in the U.S. Food and Drug Administration's Approved Drug Products with Therapeutic Equivalence Evaluations (the “Orange Book”) as corresponding to FLOMAX®.

U.S. Pat. No. 4,772,475 (EP 194838, EP 533297) discloses controlled-release pharmaceutical dosage forms comprising multiple granulate units containing tamsulosin, microcrystalline cellulose and a release control agent. The granulate gradually releases tamsulosin from the granulate matrix. The patent suggests that an enteric coating is not needed.

The disclosed process for producing the granulate units comprises granulating a mixture of tamsulosin, an unit-forming inert material such as microcrystalline cellulose and a release controlling agent comprising water and/or an aqueous emulsion, suspension or gel of a water-insoluble macromolecular substance or a solution of said macromolecular substance in an aqueous organic solvent. The macromolecular substance is preferably selected from a range of acrylic polymers, commercially sold under brand name Eudragit®. The release controlling agent serves essentially also as a binder in the granulation process. The resulting granulate may be used for making final dosage forms, capsules as well as tablets.

Example 1 of U.S. Pat. No. 4,772,475 illustrates the process. After sufficiently mixing 5 g tamsulosin HCl and 470 g microcrystalline cellulose, a mixture of 83.3 g (25 g as solid component) of Eudragit L 30 D-55 and 500 g of water was added thereto and the resultant mixture was granulated by a high-speed mixer. The granules obtained were spheres having particle sizes of 0.1 to 1.5 mm, mainly 0.2 to 1.0 mm.

U.S. Pat. No. 4,772,475 also discloses that pellets of various compositions were prepared and tested for release characteristics according to standardized Pharmacopoeial method (paddle, 150 rpm). The reported results show that in one hour in simulated gastric fluid the release ranged from 16.2 to 60.4% of the active compound. Tablets made from some of the produced pellets, having 50.3 and 57.6% release, respectively, were also tested on human volunteers in comparison with conventional tablets and concentration of the active substance in blood plasma was measured. Peak plasma levels were reached 3 hours after ingestion (in comparison with 2 hours at conventional tablets), the total amount of tamsulosin in plasma being about 75% of that of the conventional tablet.

However, such release rate is generally not sufficiently for an extended-release dosage form. It would be desirable to provide an alternative, coated tamsulosin pellet having good release characteristics.

### SUMMARY OF THE INVENTION

The present invention relates to a pharmaceutical pellet composition comprising tamsulosin as an active ingredient and having an advantageous coating layer with respect to obtaining an extended release profile. Accordingly, a first aspect of the present invention relates to a pharmaceutical dosage form comprising a plurality of pellets. Each pellet comprises a pellet core, which has a diameter within the range of 0.1-1.5 mm, preferably 0.3-0.9 mm, comprising tamsulosin or a pharmaceutically acceptable salt thereof, preferably tamsulosin hydrochloride, an inert pellet-forming carrier, preferably microcrystalline cellulose, a release control agent, preferably water permeable acrylic polymer, and optionally water. Each pellet core is surrounded by an outer layer coat, which comprises a pharmaceutically acceptable acid-resistant polymer, preferably an acid-resistant acrylic polymer, in an amount, calculated on a dry pellet core basis, that is within the range of 1 to 25 mass %. The plurality of pellets exhibits a dissolution release profile in simulated gastric fluid using Ph. Eur. basket method at 100 r.p.m which includes releasing less than 25% of the tamsulosin during the first two hours. Preferably the pellet core contains from 0.05 to 5.0% of the tamsulosin or salt thereof, expressed in terms of the equivalent amount of tamsulosin hydrochloride, 50-95% of the pellet forming carrier, from 1 to 25% and preferably from 2 to 10% of the release control agent, and 2-10% water, more preferably 2.5-5% water, all calculated on a dry pellet core basis. The mass of the outer layer coat is preferably within the range of 2.5 to 15%, more preferably 8-12%, calculated on a dry pellet core basis. All percentages are in mass %.

Another aspect of the present invention relates to a process for making the above pellets, which comprises granulating a mixture of tamsulosin or a pharmaceutically acceptable salt thereof (hydrochloride salt being preferred), pellet forming carrier (microcrystalline cellulose being preferred), release control agent (acrylic polymer being preferred), a granulation liquid comprising water, and optionally auxiliary ingredients to form wet pellet cores, drying the wet pellet cores (preferably to a residual amount of water of 2-10% and most preferably to an amount of 2.5-5%), selecting the dried pellet cores (preferably by sieving) to obtain a fraction within the size range of 0.1-1.5 mm (preferably 0.3-0.9 mm), coating the selected dried pellet cores with a coating composition that comprises an acid-resistant polymer (preferably acrylic polymer), and drying the coated pellets, wherein the coating step is sufficient to provide the dried coated pellets with 1-25 mass %, preferably 2.5-15 mass % of the coating composition, calculated on the dry pellet core basis.

A further aspect of the invention relates to a process for making pharmaceutical dosage forms which comprises granulating a mixture of tamsulosin or a pharmaceutically acceptable salt thereof (hydrochloride salt being preferred), pellet forming carrier (microcrystalline cellulose being preferred), release control agent (acrylic polymer being preferred), a granulation liquid comprising water, and optionally auxiliary ingredients to form wet pellet cores, drying the wet pellet cores (preferably to a residual amount of water of 2-10% and most preferably to an amount of 2.5-5%), selecting the dried pellet cores (preferably by sieving) to obtain a fraction within the size range of 0.1-1.5 mm (preferably 0.3-0.9 mm), coating the selected dried pellet cores with a coating composition that comprises an acid-resistant polymer (preferably acrylic polymer), drying the coated pellets, testing a sample of the dried coated pellets for dissolution rate in a simulated gastric fluid, and repeating, if necessary, the coating process on the remaining dried coated pellets until a desired release rate is achieved in the testing step. In this way, an appropriate amount of outer coat layer for a given pellet core composition, pellet core size, and outer coat layer composition can be readily determined.

### DETAILED DESCRIPTION OF THE INVENTION

It has been discovered that an effective, modified release coated tamsulosin pellet composition can be formed that exhibits a dissolution release profile, when measured as a plurality of pellets, wherein less than 25% of tamsulosin, preferably less than 15% of tamsulosin and most preferably less than 10% of tamsulosin is released during the first two hours in simulated gastric fluid in basket apparatus at 100 rpm, by controlling, inter alia, the amount of coating on the pellet. Accordingly, once the coated pellets of the present invention are ingested, tamsulosin is released into the body at a rate that is characterized by minimizing the release during the pellets' residence time in the stomach environment. More advantageously, the pellet core size and composition as well as the material and amount of the coating are so selected that the resulting coated collection of pellets exhibits at least one of the following release rates in simulated intestinal fluid (sometimes referred to herein as phosphate buffer of pH 6.8), using Ph. Eur. basket method at 100 rpm: 10-50% and preferably 15-45% of the tamsulosin released in 30 minutes, 25-75%, and preferably 30-65% of the tamsulosin released in one hour, and more than 70%, preferably more than 80% of the tamsulosin released in five hours. More preferably, the pellets satisfy all three release rates.

For clarity sake, the composition of simulated gastric fluid (SGF) and simulated intestinal fluid (SIF), although well known in the art as standard solutions, are set forth below:

SGF (USP Simulated Gastric Fluid without pepsin) composition:

| | |
|---|---|
| HCl | qs pH 1.2 |
| NaCl | 0.2% |
| water | qs 1000 ml |

SIF (USP Simulated Intestinal Fluid without pancreatin) composition:

| | |
|---|---|
| KH₂PO₄ | 6.8 g |
| NaOH | qs pH 6.8 |
| water | qs 1000 ml |

The pellets of the present invention include a pellet core having a diameter within the range of 0.1 to 1.5 mm, preferably 0.3-0.9 mm, which comprises tamsulosin or a pharmaceutically acceptable salt thereof, a pellet-forming carrier, a release control agent and, optionally, water.

The tamsulosin free base can be used per se but generally a pharmaceutically acceptable salt thereof is used. Examples of tamsulosin salts include tamsulosin hydrochloride, hydrobromide, sulfate, phosphate, acetate, propionate, maleate, fumarate, malonate, lactate, citrate, tartrate, mesylate, and besylate. The most preferred is tamsulosin hydrochloride. The amount of tamsulosin and/or salt thereof is generally equivalent to 0.05 to 5.0 mass % of tamsulosin hydrochloride, based on the total mass of the dried core. The mass is equivalent to tamsulosin hydrochloride in that the mass represents an equimolar amount of tamsulosin hydrochloride to the actual moles of tamsulosin or salt thereof in the core; hence the mass of an equivalent amount of tamsulosin hydrochloride expressed as a percent.

The pellet-forming carrier is an inert material that is able to bind active ingredient and other excipients into an essentially spherical particulate material that is commonly referred to in pharmaceutical practice as a pellet. Examples of pellet-forming carriers include microcrystalline cellulose (crystalline cellulose in other terminology), alpha lactose, dextrin, mannitol, and chitosan, alone or in combinations. In the preferred composition of the pellet core microcrystalline cellulose serves as a suitable inert carrier. Preferred amounts of the pellet-forming carrier(s) are within the range of 50-95 mass %, calculated on a dry pellet core basis.

The release control agent is an excipient which allows the release of the active substance from the composition only under certain environmental conditions and/or by a certain release rate. Many of release agents are known in the art. Within the invention, the preferred agent is a pharmaceutically acceptable polymer, most preferably a water permeable polymer, used alone or in combination with other release agents. Examples of water permeable polymers include various types of acrylic polymers, and/or cellulose derivatives such as ethyl cellulose, hydroxypropylmethylcellulose and modified analogues. The release control agent(s) are generally contained in an amount from 1-25 mass %, preferably 2 to 10 mass %, calculated on a dry pellet core basis.

Preferably the release control agent is and/or comprises an acrylic polymer. Within the invention, an “acrylic polymer” means a pharmaceutically acceptable polymer of acrylic acid, such as sold under brand name CARBOPOL, or a copolymer of methacrylic acid and/or an acrylic or methacrylic acid ester, such as sold under brand name EUDRAGIT. Such compounds are, e.g., defined in Handbook of Pharmaceutical excipients, edited by A. H. Kibbe, Pharmaceutical Press London, 3^{rd} ed. (2000). The release of the active substance from the admixture with such acrylic polymers may or may not be dependent on the environmental pH. A preferred acrylic polymer is of the EUDRAGIT L series. The amount of acrylic polymer is preferably within the range of 2-25 mass %, more preferably 2.5 to 10 mass %, including about 5 mass %, calculated on a dry pellet core basis.

The acrylic polymer in the core serves as a binder and a release-controlling agent. Preferably, the polymer is an acid-resistant acrylic polymer, which releases tamsulosin dependent upon the pH. Such polymers include EUDRAGIT L products, especially EUDRAGIT L 30 D. EUDRAGIT L 30 D-55 is available as a 30% (m/V) aqueous dispersion of the acrylate polymer containing also polysorbate 80 and sodium lauryl sulphate as emulsifiers.

Alternatively, two types of release control agents may be combined together in order to induce both time-dependent and pH-dependent control of the release of tamsulosin. Use of agents that release the active substance independently of environmental pH prevents a dose dumping after the pellet core surface comes into contact with the body fluid, while agents releasing the active substance pH-dependently focus the release of a main portion of the active component into the desired part of gastrointestinal tract. An example of a polymer that releases substances independently of the pH is hydroxypropyl methylcellulose.

The pellet core typically comprises 0.05-5.0% mass of tamsulosin or its salt (e.g. tamsulosin hydrochloride), 50-95% mass of microcrystalline cellulose, 1-25%, preferably 2.5-10%, more preferably 5%, mass of the acrylic polymer, 2-10%, preferably 2.5-5%, mass of water, and 0-25%, preferably 0.5-25%, mass of other pharmaceutically acceptable excipients, calculated on the total mass of the dried core. As used herein the “dried core” means a core that has been substantially dried and has a residual solvent content from the production thereof of 15% or less, more preferably 10% or less.

Granulation is typically performed in the presence of a granulation liquid, which comprises water. Water is the most suitable solvent and/or granulation liquid in the process of pellet formation; however it is almost completely removed afterwards. It is nevertheless important that water is preferably present in the dried composition of the core as it affects, sometimes significantly, the rate of diffusion once the coating has been dissolved in the intestinal fluid. Hence, the pellet core preferably requires water to remain in the dried cores, in an amount from to 2 to 10 mass %, and preferably from 2.5 to 5 mass %, calculated on a dry pellet core basis.

The “other” pharmaceutically acceptable excipients, if present, are generally used to provide proper characteristics of the composition within the pelletization procedure and include, inter alia, plasticizers (e.g. triethylcitrate) or an anti-sticking agent (e.g. talc).

Additionally, the pellets of the present invention comprise an outer layer coat surrounding the pellet core, which comprises a pharmaceutically acceptable acid-resistant material, preferably an acid-resistant acrylic polymer, wherein the mass of said outer layer coat, calculated on a dry pellet core basis, is within the range of 1-25%, preferably 2.5-15%. The amount of gastro-resistant coating, for instance the coating based on acid-resistant acrylic polymers, depends on the size of the pellet core to be coated. For example, the smaller the size of the pellets, the more coating that is needed. Moreover, the smaller the pellet size, the more difficult it is to maintain uniformity of coating in a production batch. The 0.1 to 1.5 mm and preferably 0.3 to 0.9 mm pellet core size range of the present invention is thus advantageous with respect to obtaining the desired release profile, for coating homogeneity, and for filling into a final unit dose (e.g., capsule) with desired content homogeneity. For the pellet core size defined within our invention, it has been determined that the amount of outer layer coat should be within the above recited range. Preferably, the amount of the applied coating composition, calculated on dry basis, is between 8-12% (w/w) of the weight of the dried pellet core.

The coating material comprises a pharmaceutically acceptable acid-resistant polymer. Such polymer essentially protects the pellet core towards contact with gastric fluid and thus it minimizes the amount of tamsulosin that may be released in the stomach. Preferred coating materials comprise an acid resistant acrylic polymer. The “acid-resistant acrylic polymer” is a specific kind of the above acrylic polymer having free carboxyl groups. Such polymers are not soluble in acidic aqueous medium, while they are soluble in neutral or basic aqueous medium. Preferred acid resistant acrylic polymers include the EUDRAGIT L series, such as EUDRAGIT L 30 D-55. This acrylic polymer is available as an aqueous suspension, also comprising a small amount of emulsifiers, and may be directly used for coating in suitable coating equipment. In a particular aspect of the invention, the “acrylic polymer” used for the manufacturing of pellet core is advantageously identical with the “acid-resistant acrylic polymer” of the pellet coating. The outer layer may comprise, either alternatively or in combination with the acid-resistant acrylic a polymer, other acid resistant polymers such as cellulose acetate, cellulose acetate phthalate, hydroxypropylmethyl cellulose phthalate, hydroxypropylmethylcellulose acetate succinate etc. In addition, the coating composition may comprise other pharmaceutically acceptable excipients. For example, an anti-sticking agent, such as talc, may be added to the coating composition to avoid stickiness of the coated granules during processing. Similarly, a plasticizer such as triethylcitrate can improve the characteristics of the final film coat.

The amount of acid resistant polymer, particularly the acrylic polymer, in the coating layer is preferably within the range of 25-95 mass %, more preferably 30 to 75%, and typically 50 to 75%, calculated on a dry basis of the coating layer. Preferably, the acrylic polymer is the only acid-resistant polymer in the outer layer coat. The remainder of the outer coat layer is pharmaceutically acceptable excipients and/or other acid-resistant polymer(s) as described above.

The pellet cores of the present invention can be made by various known techniques. The main techniques are, e.g. high shear pelletization, fluid bed pelletization, hot-melt and extrusion-spheronization. Suitable equipment for producing pellet cores for the product of the invention comprise high-shear mixer/granulators, such as equipment sold by the Bohle company under the brand name Vagumator (VMA). The VMA is a single-pot system, which combines blending, wet granulation/pelletization and the subsequent drying of solid products in one piece of equipment. Blending and mixing is facilitated by the presence of high shear mixing equipment (impeller and chopper), whilst the drying process is facilitated by the presence of a microwave, nitrogen drying, vacuum drying and a heatable jacketed process vessel wall. Alternate pelletization techniques, as are known in the art, are also suitable.

A granulation method for making the pellets preferably comprises:
a. granulating a mixture of tamsulosin salt (preferably a water soluble tamsulosin salt such as tamsulosin hydrochloride), a pellet forming carrier (for instance microcrystalline cellulose), a release control agent (preferably a pharmaceutically acceptable polymer, e.g. an acrylic polymer), a granulation liquid comprising, and preferably consisting of water, and optionally auxiliary ingredients to form wet pellet cores;
b. drying said wet pellet cores, preferably to a residual amount of water of 2-10%;
c. selecting, for instance by sieving, said dried pellet cores to obtain a fraction within the size range of 0.1-1.5 mm, preferably 0.3-0.9 mm;
d. coating said sieved dried pellet cores with a coating composition that comprises an acid-resistant polymer; and
e. drying said coated pellet.

The coating step (d) is sufficient to provide the dried coated pellet with 1-25 mass %, preferably 2.5-15 mass % of said coating composition, calculated on the dry pellet core basis. Preferred acid resistant polymer is an acid resistant acrylic polymer. The auxiliary ingredients, which are pharmaceutically acceptable excipients, are typically a lubricant or plasticizer, but are not limited thereto.

A preferred granulating process may be illustrated as follows: mixing tamsulosin hydrochloride with microcrystalline cellulose and an anti-sticking agent to form a powder blend, adding a suspension of acrylic polymer and plasticizer in water to the powder blend mixture, granulating the mixture, drying the obtained granules under control of amount of residual water, and sieving the granules to proper size fractions. The drying process may be performed in the granulator or outside in an appropriate dryer. The control of the residual water content in produced pellets may be made, for example, by taking samples of pellets and annealing them in an oven at 105° C., while measuring the weight loss.

The process of coating may be performed in any suitable equipment such as, directly in the high shear mixer/granulator, in a fluid bed coater, or preferably on a coating pan. The results of the coating procedure may be routinely checked by withdrawing a sample of the pellets and determining the release rate of tamsulosin in simulated gastric fluid as described above. However, if the desired amount of release is not achieved, the coating process of the remaining coated pellets may be repeated until the desired result is obtained. The use of sampling and re-coating is a useful process for determining the amount of coating for a given core size, core composition, and coating composition to achieve the desired release rate. This process can be carried out on any amount of coating and is not necessarily limited to the 1-25 mass % coating amount. Also it is possible to mix various sub-lots of coated pellets with different release rates to obtain a final lot exhibiting the desired rate. If one sub-lot does not yield the desired pellet size distribution, the negative effects can be made up with other sub-lots.

Once the coated pellets have been produced they may be formulated into individual dosage units for administration of tamsulosin for therapeutic and/or prophylactic purposes such as capsules or sachets. Accordingly, the unit dosage forms containing pellets preferably contain tamsulosin or a salt thereof in an amount equivalent to between 0.01 to 10 mg of tamsulosin hydrochloride per unit, preferably equivalent to 0.1 to 1 mg of tamsulosin hydrochloride per unit, even more preferably equivalent to 0.2, 0.4 or 0.8 mg of tamsulosin hydrochloride per unit. Such a unit dose is normally taken from 1 to 3 times daily, preferably once a day. In practice, the physician will determine the actual dosage and administration regimen, which will be the most suitable for the individual patient.

The suitable unit dosage form may comprise pharmaceutically acceptable capsules of a suitable size (e.g. No. 2 size), for example made from hard gelatin or hydroxypropyl methylcellulose. These coated pellets display an excellent flowability and content uniformity.

Capsules with coated pellets of the present invention comprising a unit dosage amount of tamsulosin may be delivered for immediate use in a suitable package comprising advantageously from 5 to 100 capsules. Such package may comprise a blister pack comprising advantageously 10, 14, 20, 28 or 30 capsules, or a plastic or glass container/bottle containing the same amounts of capsules. Any suitable pharmaceutically acceptable package material may be used in production the package unit.

Coated pellets for oral administration of tamsulosin according to the present invention may be used, for example, in the management of functional treatment of symptomatic benign prostatic hypertrophy or hyperplasia (BPH) or other disorders treatable by tamsulosin (the Disorders). The gastro-resistant coating and extended release of tamsulosin from pellet core assures that therapeutic concentration of tamsulosin in blood is maintained for sufficiently long time, without initial dumping in the stomach.

Accordingly, the present invention further provides a method for treating and/or preventing any one or more disorders which comprises orally administering an effective and/or prophylactic amount to a sufferer in need thereof, of tamsulosin or its pharmaceutically acceptable acid addition salt, particularly tamsulosin hydrochloride, which is formulated into a coated pellet comprising the composition as specified above. Preferably, the pellets of the invention are administered once a day and more preferably after meal. Administration after food intake is advantageous because of better dispersion of pellets in the environment and minimizing damages of tissues of gastrointestinal tract.

The present invention also provides the use of the tamsulosin pellet comprising the composition as specified above, as well as the use of the above process for making the tamsulosin pellet composition itself, for the manufacture of a medicament for treating and/or preventing any one or more of the Disorders. Also, the coated pellets may be used in medical applications in combination with other agents. The combination may be realized in a form of single combination preparation or by separate administration of drugs containing the above agents.

The invention is further illustrated by the following Examples, but should not be construed as being limited thereto.

### EXAMPLE 1

### Tamsulosin Hydrochloride 0.4 mg Enteric-Resistant Pellets

Formula Used:

| Ingredients | g per batch |
|---|---|
| pellet core: | |
| Tamsulosin.HCl | 2.9 |
| Eudragit L 30 D-55 | 401.1 |
| Triethylcitrate | 12.2 |
| Talc | 120.2 |
| Microcrystalline cellulose | 2000.4 |
| Water (demineralized) | 2000.0 |
| pellet coating (1000 g of pellets) | |
| Eudragit L 30 D-55 (30% dispersion) | 166.48 |
| Triethylcitrate | 5.0 |
| Calcium stearate | 10.0 |
| Water (demineralized) | 106.0 |

Manufacturing Process:

High shear mixer/granulator VMA 10 was used.
Tamsulosin hydrochloride was mixed with talc and microcrystalline cellulose to a homogeneous powder blend
A suspension of Eudragit, triethyl citrate and water was prepared in a separate vessel
The suspension was added to the powder blend and the mixture was granulated.
The produced granulate was dried by vacuum, nitrogen and microwave until the moisture content of the pellets was 2.7%.
The dried granulate was sieved and fractions between 0.3 and 0.85 mm were collected.

The Pellet Size Distribution:

| particle size (mm) | g | % |
|---|---|---|
| x > 1.0 | 307.0 | 14.3 |
| 0.85 < x < 1.0 | 44.0 | 2.0 |
| 0.6 < x < 0.85 | 767.1 | 35.7 |
| 0.5 < x < 0.6 | 857.4 | 39.9 |
| 0.425 < x < 0.5 | 84.4 | 3.9 |
| 0.3 < x < 0.425 | 67.6 | 3.1 |
| x < 0.3 | 20.0 | 0.9 |
| total batch | 2147.5 | 100.0 |

Pellet coating process: 1000 g of the proper sized pellets were returned to the VMA 10. The coating was applied at a rate of ±8 ml/min. The coating was applied in 60 minutes. After drying for 1.5 hours, the batch was discharged and samples were taken for examination.
Pellet Coating Results:

The content of residual water, measured by moisture analyzer, of the coated pellets was 2.8%. Weight gain after coating: 6.5%.

The dissolution profile in simulated gastric fluid: less than 5% in 2 hours.

The dissolution profile in pH 6.8 buffer (SIF): 20% in 30 minutes, 35% in 1 hour, 90% in 5 hours.

### EXAMPLE 2

Formula Used:

| Ingredients | total weight (g) | dry weight (%) |
|---|---|---|
| pellet core: | | |
| Tamsulosin.HCl | 20.23 | 20.23 |
| Eudragit L 30 D-55 | 2780.75 | 834.33 |
| Triethylcitrate | 83.44 | 83.44 |
| Talc | 834.23 | 834.23 |
| Microcrystalline cellulose | 14000.00 | 14000.00 |
| Water (demineralized) | 14000.00 | |
| Pellet coating (of 13.8 kg pellets) | | |
| Eudragit L 30 D-55 (30% disp.) | 4600 | 1380 |
| Talc | 552 | 552 |
| Triethylcitrate | 138 | 138 |
| Water | 5066 | |

The solids content of this coating suspension is 20.2% (including the triethylcitrate, this is a liquid but it will not evaporate during coating)

Manufacturing Process:

As in Example 1. High shear mixer/granulator VMA 70 was used.

Results. Yield in pellets of proper size: 13823 g=84.7%.

The content of residual water of the pellets was 3.4%.

Pellet Size Before Coating:

| particle size (mm) | g | % |
|---|---|---|
| x ≧ 0.85 | 0.9 | 1.1 |
| 0.5 < x < 0.85 | 18.7 | 22.1 |
| 0.425 < x < 0.5 | 45.7 | 54.0 |
| 0.3 < x < 0.425 | 16.8 | 19.9 |
| x < 0.3 | 2.5 | 3.0 |
| total sample | 84.6 | 100.0 |

Pellet coating: The pellet coating was performed with a 25 l solid pan.

Samples at levels of 8, 9, 10, 11 & 12% coating were taken during production for determination of the dissolution profile in SGF.

Results:

Pellet size distribution after coating:

| particle size (mm) | g | % |
|---|---|---|
| x ≧ 0.85 | 14.7 | 3.0 |
| 0.6 < x < 0.85 | 254.3 | 51.1 |
| 0.5 < x < 0.6 | 117.2 | 23.6 |
| 0.425 < x < 0.5 | 100.0 | 20.1 |
| 0.3 < x < 0.425 | 11.1 | 2.2 |
| x < 0.3 | 0.1 | 0.0 |
| total sample | 497.4 | 100.0 |

Dissolution Results:

The dissolution profiles in SGF, basket, 100 rpm gave the following results: at least 10% of coating should be applied to these pellets to match the required dissolution profile in SGF.

The dissolution profile of the made coated pellets in phosphate buffer (basket method, 100 rpm, pH=6.8): 41% in 30 minutes, 59% in 1 hour, 99% in 300 minutes.

All of the patents, articles, and documents mentioned above are incorporated herein by reference in their entirety. In addition, the disclosure of co-pending U.S. patent application Ser. No. 10/293,940, which describes some preferred embodiments of the present invention, is incorporated herein in its entirety. The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

1. A pharmaceutical dosage form comprising a plurality of pellets, wherein each pellet comprises:
a. a pellet core having a diameter within the range of 0.1 to 1.5 mm and comprising tamsulosin or a pharmaceutically acceptable salt thereof, an inert pellet forming carrier, a release control agent and optionally water; and
b. an outer layer coat surrounding said core which comprises a pharmaceutically acceptable acid-resistant polymer, wherein the mass of said outer layer coat, calculated on a dry pellet core basis, is within the range of 1-25%; and
wherein the plurality of pellets exhibits a dissolution release profile in simulated gastric fluid using Ph. Eur. basket method at 100 rpm which includes releasing less than 25% of the tamsulosin or salt thereof during the first two hours.

2. The dosage form according to claim 1, wherein the tamsulosin is a salt selected from the group consisting of tamsulosin hydrochloride, hydrobromide, sulfate, phosphate, acetate, propionate, maleate, fumarate, malonate, lactate, citrate, tartrate, mesylate, and besylate.

3. The dosage form according to claim 2, wherein the tamsulosin salt is tamsulosin hydrochloride.

4. The dosage form according to claim 1, wherein the amount of the tamsulosin or salt thereof in the pellet core is equivalent to 0.05-5.0 mass % of tamsulosin hydrochloride, calculated on a dry pellet core basis.

5. The dosage form according to claim 1, wherein the pellet forming carrier comprises a material selected from the group consisting of microcrystalline cellulose, alpha lactose, dextrin, mannitol, chitosan, and combinations thereof.

6. The dosage form according to claim 5, wherein said pellet forming carrier is microcrystalline cellulose.

7. The dosage form according to claim 5, wherein the amount of the pellet forming carrier is 50-95 mass %, calculated on a dry pellet core basis.

8. The dosage form according to claim 1, wherein the release control agent comprises a pharmaceutically acceptable polymer.

9. The dosage form according to claim 8, wherein the pharmaceutically acceptable polymer is selected from the group consisting of acrylic polymers, cellulose derivatives, and combinations thereof.

10. The dosage form according to claim 9, wherein the pharmaceutically acceptable polymer is a water permeable acrylic polymer.

11. The dosage form according to claim 8, wherein the amount of the release control agent is from 1-25 mass %, calculated on a dry pellet core basis.

12. The dosage form according to claim 1, wherein the content of water in the pellet core is from 2 to 10%, calculated-on a dry pellet core basis.

13. The dosage form according to claim 1, wherein the diameter of the dried pellet core is within the range from 0.3 to 0.9 mm

14. The dosage form according to claim 1, wherein the acid-resistant polymer comprises an acid-resistant acrylic polymer.

15. The dosage form according to claim 14, wherein the release control agent in the pellet core is the same as the acid-resistant acrylic polymer in the outer coat.

16. The dosage form according to claim 14, wherein the outer layer coat comprises 25-75 mass % of the acid-resistant acrylic polymer, calculated on a dry basis.

17. The dosage form according to claim 1, wherein said mass of the outer layer coat, calculated on a dry pellet core basis, is within the range of 2.5-15 mass %.

18. The dosage form according to claim 1, wherein the dissolution release profile in simulated gastric fluid includes releasing less than 15% of tamsulosin during the first two hours in Ph. Eur. basket apparatus at 100 rpm.

19. The dosage form according to claim 1, wherein the plurality of pellets exhibit a dissolution release profile in a phosphate buffer of pH 6.8 using Ph. Eur. basket method at 100 rpm which includes releasing:
10-50% of the tamsulosin in 30 minutes, and/or
25-75% of the tamsulosin in one hour, and/or
more than 70% of the tamsulosin in five hours.

20. The dosage form according to claim 1, wherein the dosage form is a capsule or sachet.

21. The dosage form according to claim 20, wherein the amount of tamsulosin or salt thereof contained in the pellet is equivalent to 0.1 to 1 mg of tamsulosin hydrochloride.

22. A process for making the dosage form according to claim 1, which comprises:
a. granulating a mixture of tamsulosin or a pharmaceutically acceptable salt thereof, a pellet forming carrier, a release control agent, a granulation liquid comprising water, and optionally auxiliary ingredients to form wet pellet cores;
b. drying said wet pellet cores;
c. selecting said dried pellet cores to obtain a fraction within the size range of 0.1-1.5 mm;
d. coating said selected dried pellet cores with a coating composition, which comprises an acid-resistant polymer and which is sufficient to provide said dried coated pellet with 1 to 25 mass % of said coating composition, calculated on the dry pellet core basis; and
e. drying said coated pellet.

23. The process according to claim 22, wherein the tamsulosin is a salt selected from the group consisting of tamsulosin hydrochloride, hydrobromide, sulfate, phosphate, acetate, propionate, maleate, fumarate, malonate, lactate, citrate, tartrate, mesylate, and besylate.

24. The process according to claim 23, wherein the amount of the tamsulosin salt in the pellet core, is equivalent to 0.05-5.0 mass % of tamsulosin hydrochloride, calculated on a dry pellet core basis.

25. The process according to claims 22, wherein the pellet forming carrier comprises a material selected from the group consisting of microcrystalline cellulose, alpha lactose, dextrin, mannitol, chitosan and combination thereof.

26. The process according to claim 22, wherein the pharmaceutically acceptable polymer is a water permeable acrylic polymer.

27. The process according to claim 22, wherein the content of water in the pellet core after drying is from 2 to 10%, calculated on a dry pellet core basis.

28. The process according to claim 22, wherein the selection of the pellet core fraction is performed by sieving.

29. The process according to claim 28, wherein the diameter of the dried pellet core is within the range from 0.3 to 0.9 mm.

30. The process according to claim 22, wherein the mass of the outer layer coat, calculated on a dry pellet core basis, is within the range of 2.5-15 mass %.

31. The process according to claim 22, wherein said coating step (d) is performed in a high shear mixer/granulator.

32. The process according to claim 22, wherein said coating step (d) is performed in a fluid bed coater.

33. The process according to claim 22, wherein said coating step (d) is performed on a coating pan.

34. A process for making a pharmaceutical dosage form, which comprises the steps of:
a. granulating a mixture of tamsulosin or a pharmaceutically acceptable salt thereof, a pellet forming carrier, a release control agent, a granulation liquid comprising water, and optionally auxiliary ingredients to form wet pellet cores;
b. drying said wet pellet cores;
c. selecting said dried pellet cores to obtain a fraction within the size range of 0.1-1.5 mm;
d. coating said selected dried pellet cores with a coating composition, which comprises an acid-resistant polymer;
e. drying said coated pellet;
f. testing a sample of said dried coated pellets for dissolution rate in a simulated gastric fluid; and
(g) repeating the coating process on the remaining dried coated pellets until a desired rate of release is achieved in said testing step (f).

35. The process according to claim 34, wherein the desired rate of release includes less than 25% of the tamsulosin during the first two hours.

36. A method for treating the symptoms of benign prostatic hyperplasia, which comprises administering an effective amount of the pellets according to claim 1, to a patient in need thereof.
